(19) United States
(12) Patent Application Publication
(10) Pub. No.: US 2021/0402198 A1
(43) Pub. Date: Dec. 30, 2021

(54) PROSTATE AND HEMORRHOID TREATMENT DEVICE

(71) Applicant: THE CATHOLIC UNIVERSITY OF KOREA INDUSTRY-ACADEMIC COOPERATION FOUNDATION, Seoul (KR)

(72) Inventors: Dong Won MOON, Seoul (KR); Sung Hee MOON, Seoul (KR); Woong Jin BAE, Gyeonggi-do (KR)

(21) Appl. No.: 17/288,418

(22) PCT Filed: Dec. 28, 2018
(86) PCT No.: PCT/KR2018/016943

§ 371 Date: Apr. 23, 2021

(30) Foreign Application Priority Data
Oct. 26, 2018 (KR) 10-2018-0129033

(51) Int. Cl. A61N 2/00; A61N 5/06; A61F 7/12; A61N 1/20; A61N 1/05; A61N 1/08; A61H 21/00

(52) U.S. Cl. CPC A61N 2/002; A61N 5/0613; A61F 7/12; A61N 1/205; A61F 2007/0028; A61N 1/08; A61H 21/00; A61N 2005/0661; A61N 2005/0608; A61N 1/0512

(57) ABSTRACT

A device for treating the prostate and hemorrhoids is proposed, the device including: an anal insertion part including a body module including a first rechargeable battery charged when DC power is applied therein, a first wireless communication device wirelessly transmitting and receiving an electric signal, and an output circuit outputting a current according to the electric signal output from the first wireless communication device, a plurality of electrodes provided in a direction perpendicular to the body module and installed apart along a longitudinal direction on an outer circumferential surface to receive the current applied thereto, and a probe module including a vibrating element installed therein to generate vibration when the current is applied, a heating element generating heat, and a magnet installed at an upper end therein to generate a magnetic force; and a controller provided with a second rechargeable battery, a second wireless communication device, and a control module.

### BACKGROUND

The present invention belongs to a technical field of medical devices. In particular, among medical devices, the present invention belongs to the technical field of a device for treating the prostate or hemorrhoids by inserting the device into the anus.

Together with the testicles, the prostate is one of the reproductive organs that enable reproduction. The prostate makes and secretes some of liquid components that makes up semen. The prostate produces prostate fluid, which is equivalent to about 35% of the semen.

Prostate fluid nourishes sperm and enables the ejaculated semen not to be clotted, thereby enhancing motility of sperm. In addition, the prostate fluid is alkaline and neutralizes strong acidity of the fallopian tube so as to enable sperm to safely pass through the fallopian tube and meet with the egg for fertilization. That is, the prostate fluid increases sperm fertility. The prostate fluid, which increases fertility, is produced by a strong prostate.

A healthy prostate may be maintained in a proper lifestyle of a man. However, a large number of men in their 30s in modern society drink alcohol and smoke, resulting in deteriorating prostate health. Thus, many men in their 30s suffer from an enlarged prostate as an example of prostate disease. The medical industry is actively developing devices that are capable of treating prostate diseases in order to solve the problems of men having the prostate diseases. For example, an ultrasound prostate treatment device such as the “ULTRASONIC PROSTATE THERAPEUTIC INSTRUMENT” disclosed in Korean Patent Application Publication No. 10-2001-0011952 has been developed.

However, the developed prostate treatment device has a problem in that not only an error range of heat generated by a probe thereof is large, but also a treatment range in which the prostate is treated with heat is narrow.

### SUMMARY OF THE INVENTION

The present invention may allow to massage the anus with electric stimulation, vibration, and heat, help blood circulation by magnetic force, and improve health of the prostate and anus.

The technical problems of the present invention are not limited to the above-mentioned technical problems, and other technical problems not described above will be clearly understood by those skilled in the art from the description of the claims.

In order to solve the above technical problems, the present invention provides a prostate and hemorrhoid treatment device including:

an anal insertion part including a body module including a first rechargeable battery charged when DC power is applied therein, a first wireless communication device for wirelessly transmitting and receiving an electric signal, and an output circuit for outputting a current according to the electric signal output from the first wireless communication device, a plurality of electrodes provided in a direction perpendicular to the body module and installed apart along a longitudinal direction on an outer circumferential surface to receive the current applied thereto, and a probe module including a vibrating element installed therein to generate vibration when the current is applied, a heating element for generating heat, and a magnet installed at an upper end therein to generate a magnetic force; and

a controller provided with a second rechargeable battery charged when the DC power is applied therein, a second wireless communication device capable of performing wireless communication with the first wireless communication device, and a control module for changing the electric signal output from the second wireless communication device.

The prostate and hemorrhoid treatment may further include: a converter charging part including a converter module for converting AC power applied from outside into the DC power and a plurality of accommodating space modules for vertically accommodating and then fixing the anal insertion part and the controller therein, wherein the converter charging part may charge the first rechargeable battery and the second rechargeable battery by respectively applying the DC power to the anal insertion part and the controller, which are fixed to the accommodation space module.

The controller may include at least one ultraviolet lamp installed on an outer surface thereof, and may operate the ultraviolet lamp when the second rechargeable battery is charged.

The converter charging part may be coupled to a lid part covering the anal insertion part and the controller, which are fixed to the accommodation space module,

the lid part may include a position magnet on a lower part surface in contact with the converter charging part, and

the converter charging part may include a magnetic sensor on an upper part surface in contact with the lid part, so that the converter charging part may generate a first signal when the magnetic sensor detects the position magnet and generate a second signal when charging the second rechargeable battery, thereby operating at least one ultraviolet lamp installed on an outer side surface of the controller.

The plurality of electrodes may include a lower electrode, a middle electrode, and an upper electrode, so that the lower electrode and the middle electrode, or the lower electrode and the upper electrode, or the middle electrode and the upper electrode may be able to be operated when the current is applied.

The output circuit may include a medium-frequency current output circuit for outputting a preset medium-frequency current, and each of the electrodes may include a medium-frequency electrode plate through which the medium-frequency current output from the medium-frequency current output circuit is respectively conductive.

The controller may include a mode selection module having a treatment mode including a prostate treatment mode and a hemorrhoid treatment mode, and

the control module may be able to output different electric signals from the second wireless communication device according to the treatment mode selected by the mode selection module.

In the device for treating the prostate and hemorrhoids according to the present invention, a heating element is installed only in a part of an upper part of the device, whereby a change in temperature of generated heat may be minimized.

The present invention minimizes a temperature change of heat generated from the heating element, so that the heat of a predetermined temperature may be completely transferred to the prostate. In addition, the present invention expands a treatment range by generating electric stimulation through several channels and enables accurate electrotherapy to be performed. In addition, the present invention may minimize an impact received by a patient by controlling intensity of stimulation.

In addition, the present invention generates vibration during the electrotherapy to massage inside the anus, and may help circulation of blood in the anus.

In addition, the present invention sterilizes bacteria on an anal insert part through a sterilization function performed through emitting ultraviolet rays onto itself, so as to enable the anal insert part to be safely reused.

### BRIEF DESCRIPTION OF DRAWINGS

FIG. 1 is a perspective view showing a prostate and hemorrhoid treatment device according to an exemplary embodiment of the present invention.

FIG. 2 is an assembled perspective view showing a state in which the prostate and hemorrhoid treatment device of FIG. 1 is coupled to a cover part and a converter charging part.

FIG. 3 is a view showing an internal state of the anal insertion part schematically illustrating an internal configuration of the anal insertion part of FIG. 1 and an internal state of a controller schematically illustrating an internal configuration of the controller.

FIG. 4 is a view showing an operating state of the anal insertion part operated by the controller of FIG. 1.

FIGS. 5 to 7 are views showing use states of FIG. 1.

FIG. 8 is a view showing a state in which the anal insertion part of FIG. 1 is sterilized by ultraviolet rays.

### DETAILED DESCRIPTION OF THE INVENTION

Hereinafter, exemplary embodiments of the present invention will be described in detail with reference to the accompanying drawings.

The exemplary embodiments of the present invention are provided to more completely describe the present invention to those skilled in the art, and the following exemplary embodiments may be modified in various forms. Accordingly, the scope of the present invention is not limited to the following exemplary embodiments.

In addition, the same reference numerals in the drawings of the present specification refer to the same elements.

Hereinafter, with reference to the drawings showing the exemplary embodiment of the present invention will be described.

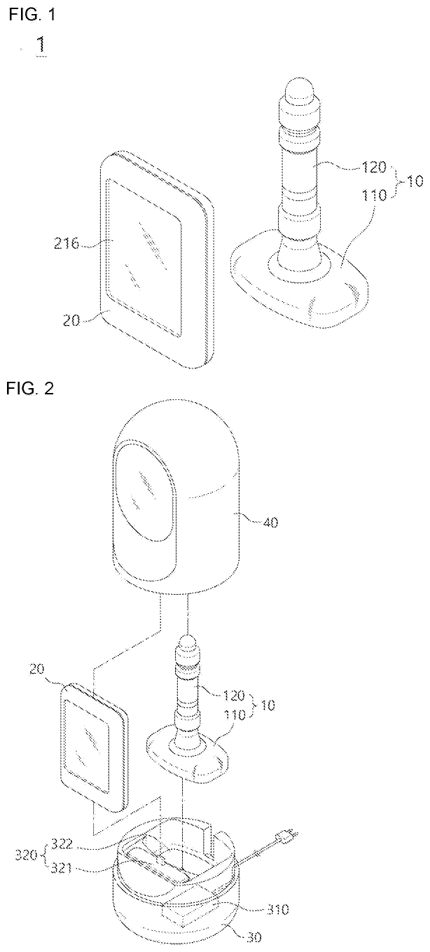

First, a device for treating the prostate and hemorrhoids according to the exemplary embodiment of the present invention will be generally described with reference to FIG. 1.

FIG. 1 is a perspective view showing a prostate and hemorrhoid treatment device according to the exemplary embodiment of the present invention.

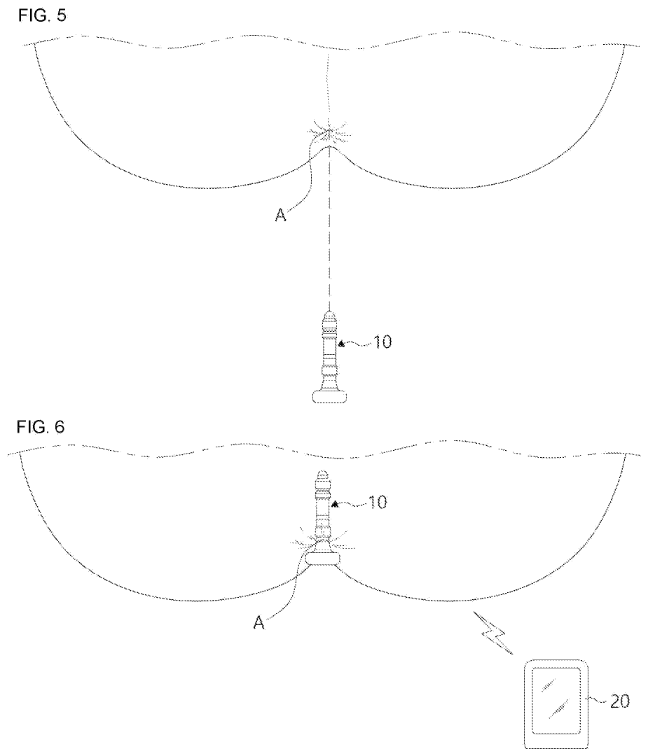

In the prostate and hemorrhoid treatment device 1 according to the exemplary embodiment of the present invention, an anal insertion part 10 is inserted into the anus of a human body, and stimulation including electricity, vibration, and heat is applied to the prostate, so that health of the prostate may be improved. In addition, the prostate and hemorrhoid treatment device 1 stimulates the anus A (see FIG. 5) with electricity, vibration, heat, and magnetic force, and helps blood circulation in the anus, so that diseases that occur in the anus, such as hemorrhoids, may be alleviated.

That is, the prostate and hemorrhoid treatment device 1 of the present invention generates electric stimulation, vibration, heat, and magnetic force after inserting the anal insertion part 10 into the anus A, so that the prostate and anus become healthy.

Such a prostate and hemorrhoid treatment device 1 may be configured to include: an anal insertion part 10 inserted into the anus A of the human body; and a controller 20 wirelessly communicating with the anal insertion part 10 and controlling an operation of the anal insertion part 10.

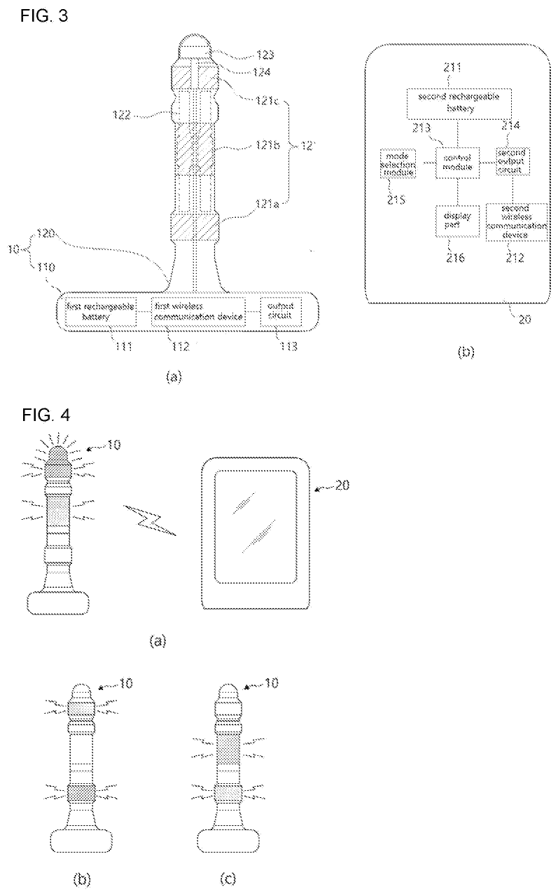

Hereinafter, components of the prostate and hemorrhoid treatment device and components coupled to the prostate and hemorrhoid treatment device will be described in detail with reference to FIGS. 2 and 3.

FIG. 2 is an assembled perspective view showing a state in which the prostate and hemorrhoid treatment device of FIG. 1 is coupled to a cover part and a converter charging part, and FIG. 3 is a view showing an internal state of the anal insertion part schematically illustrating an internal configuration of the anal insertion part of FIG. 1 and an internal state of a controller schematically illustrating an internal configuration of the controller.

The anal insertion part 10 may be configured to include: a body module 110 inserted into a second accommodation space module 322 of a converter charging part 30; and a probe module 120 inserted into the anus A to be able to contact the prostate.

The body module 110 may be formed in an elliptical sphere whose length is longer than a height thereof. However, a shape of the body module 110 is not limited to the shape of such an elliptical sphere. The shape of the body module 110 may be variously changed as long as the shape enables a user to easily grip the body module 110 and allows the body module 110 to be easily inserted into the second accommodation space module 322 of the converter charging part 30.

Inside the body module 110, a first rechargeable battery 111, a first wireless communication device 112, and an output circuit 113 may be installed. In this case, the first rechargeable battery 111 may be charged by DC power applied through the converter charging part 30 and may operate the first wireless communication device 112. While operating, the first wireless communication device 112 may transmit and receive an electric signal wirelessly and may apply the electric signal to the output circuit 113.

The output circuit 113 may apply current to various devices installed in the probe module 120 by calculating the electric signal transmitted from the first wireless communication device 112. In this case, the output circuit 113 is formed as a medium-frequency current output circuit, and may output a preset medium-frequency current.

The probe module 120 is formed in a vertical direction from a central part of the upper side of the body module 110. The probe module 120 includes the above-described various devices, that is, a plurality of electrodes, a vibrating element, a heating element, a magnet, and the like. Here, the plurality of electrodes 121 may include a lower electrode 121a, a middle electrode 121b, and an upper electrode 121c, and may be provided on an outer surface of the probe module 120 at a predetermined interval. For example, the lower electrode 121a is installed at a lower end of the probe module 120 adjacent to the body module 110, the middle electrode 121b is installed at a predetermined interval in the direction of the upper end of the probe module 120 from the lower electrode 121a, and the upper electrode 121c is installed at a predetermined interval in the direction of the upper end of the probe module 120 from the middle electrode 121b.

When the output circuit is provided as a medium-frequency current output circuit, the plurality of electrodes 121 may be provided as a medium frequency electrode plate capable of conducting a medium-frequency current output from the medium-frequency current output circuit.

In addition, an adhesive pad may be attached to the plurality of electrodes 121. Here, the adhesive pad may be a conductive adhesive pad that prevents any irritation or injury to the anus.

A vibrating element 122 may generate vibrations of different intensity depending on a magnitude of the current applied from the output circuit 113. Such a vibrating element 122 operates at the same time when electric stimulation from the plurality of electrodes 121 is generated and may massage the inside of the anus. At this time, the vibration generated by the vibrating element 122 massages the inside of the anus and helps blood circulation in the anus so that the prostate and anus become healthy. In addition, the vibration reduces a coefficient of friction between the anus and the probe module 120, so as to enable a user to remove the probe module 120 from the anus while the adhesive pad attached to the probe module 120 is not separated.

The heating element 124 may generate heat of different intensity depending on the current applied from the output circuit. Such a heating element 124 may be installed at a position between the upper electrode 121c and the uppermost end of the probe module 120. That is, the heating element 124 may be provided at an area where the probe module 120 contacts the prostate.

Such a heating element 124 may have a height thereof smaller than a distance between the upper electrode 121c and the uppermost end of the probe module 120, and may have a width shorter than the height thereof. That is, the heating element 124 may be provided in a small size inside the probe module 120. The small-sized heating element 124 generates a minimum temperature change and may deliver constant heat to the prostate.

The magnet 123 is installed between the heating element 124 and the uppermost end of the probe module 120 to generate a magnetic force. The magnet 123 may alleviate hemorrhoid disease by promoting blood circulation in the anus.

The controller 20 wirelessly communicates with the anal insertion part 10 to control the magnitude of electricity, vibration, and heat, which are generated by the anal insertion part 10. Inside the controller 20, a second rechargeable battery 211, a second wireless communication device 212, a control module 213, a second output circuit 113, a mode selection module 215, and the like may be installed. In addition, a display part 216 serving as a touch panel may be installed on one outer side surface of the controller 20, and an ultraviolet lamp 220 may be installed on the opposite side surface of the controller 20.

In this case, the second rechargeable battery 211 installed therein is charged by DC power applied through the converter charging part 30 in the same manner as the first rechargeable battery 111 described above. In addition, the second wireless communication device 212 wirelessly communicating with the first wireless communication device 112 and the control module 213 for changing an electric signal generated from the second wireless communication device 212 may be operated with the charged electric charge.

Here, the control module 213 may become a microcontroller unit, that is, an MCU, and may drive the second output circuit 113 capable of operating the second wireless communication device 212.

In addition, while connected to the mode selection module 215, the control module 213 may output a signal by calculating a signal input from the mode selection module 215.

By including a prostate treatment mode and a hemorrhoid treatment mode, the mode selection module 215 may apply different electric signals to the control module 213 according to a treatment mode selected by a user through the display part 216. For example, the mode selection module 215 may apply electric signals to the control module 213 by generating an electric signal “A” when in the prostate treatment mode and generating an electric signal “B” when in the hemorrhoid treatment mode.

By calculating different electric signals applied from the mode selection module 215 and generating electric signals corresponding to the calculated electric signals, the control module 213 may output different electric signals from the second wireless communication device 212.

The anal insertion part 10 and the controller 20 of the present invention may be charged after being inserted into the converter charging part 30 vertically and fixed thereto. the converter charging part 30 includes a converter module 310 installed therein and a plurality of accommodation space modules 320 provided outside thereof.

The converter charging part 30 converts AC power applied from the outside by the converter module 310 into DC power, and charges both the anal insertion part 10 inserted into the first accommodation space module 321 and the controller 20 inserted into the second accommodation space module 322.

In addition, the converter charging part 30 may include a magnetic sensor that is installed on a surface in contact with a lid part 40 to detect a detachable coupling with the lid part 40. In this case, the magnetic sensor may be installed at a position on a side of the power line, that is, on a rear side of the converter charging part 30, or may be installed at a position on a left side or a right side with respect to the rear side thereof. However, this installation case is only an example, and the installation position of the magnetic sensor is not limited thereto, and may be installed at various positions on the surface of the converter charging part 30.

In addition, the converter charging part 30 may include an operation signal output device for calculating and outputting, as each signal, a state of the magnetic sensor and a charging state of the anal insertion part 10 and the controller 20.

After accommodating the anal insertion part 10 and the controller 20, the converter charging part 30 is detachable from the lid part 40 and may be combined or separated from the lid part 40. In addition, the converter charging part 30 detects the attachment and detachment of the lid part 40 and may apply an electric signal to the controller 20 fixed to the accommodation space module 320.

The lid part 40 may be formed in a shape in which one side thereof is partially flat, but a longitudinal section thereof is in a shape of an arch. For example, The lid part 40 may be formed in a shape as shown in FIG. 2.

A position magnet for controlling turn-on and turn-off operations of the magnetic sensor of the converter charging part 30 may be installed on a lower part surface of the lid part 40 in contact with the converter charging part 30. Here, the position magnet may be installed on the lower part surface of the lid part that contacts the magnetic sensor when the lid part 40 contacts the converter charging part 30.

Such a lid part 40 may be coupled to the converter charging part 30 so that a side on which the flat surface is formed becomes the front side. That is, the lid part 40 may always be coupled to the converter charging part 30 in the same direction.

In the lid part 40 and the converter charging part 30, whether the lid part 40 and the converter charging part 30 are coupled to each other or not may be detected through the position magnet and the magnetic sensor.

The lid part 40 and the controller 20 are coupled with each other, so as to enable a user to conveniently carry the prostate and hemorrhoid treatment device 1, that is, the anal insertion part 10 and the controller 20. In addition, depending on whether the lid part 40 and the converter charging part 30 are coupled with each other, the controller 20 may be operated to sterilize the anal insertion part 10. A detailed description of the sterilization of the anal insertion part 10 through the controller 20 will be described later.

Hereinafter, with reference to FIG. 4, a state of controlling an operation of the anal insertion part through the controller will be described.

FIG. 4 is a view showing an operating state of the anal insertion part operated by the controller of FIG. 1.

The controller 20 transmits the electric signal output from the second wireless communication device 212 to the first wireless communication device 112 of the anal insertion part 10 and may control the operation of the anal insertion part 10. For example, as shown in FIG. 4(a), the controller 20 may allow the heating element 124 of the anal insertion part 10 to generate heat. In addition, the controller 20 may allow electric stimulation to be generated in the upper electrode 121c and the middle electrode 121b. In addition, as shown in FIG. 4(b) the controller 20 may allow electric stimulation to be generated at the upper electrode 121c and the lower electrode 121a, and as shown in FIG. 4(c), the controller 20 may allow electric stimulation to be generated in the middle electrode 121b and the lower electrode 121a.

As described above, the controller 20 may accurately apply electric stimulation to a specific treatment site by allowing the anal insertion part 10 to generate electric stimulation through multiple channels. Moreover, through the second output circuit, the intensity that each patient may feel as pain may be minimized by changing a magnitude of the electric signal received by the first wireless communication device 112 to adjust the intensity of stimulation generated from the electrodes.

Hereinafter, use states of the prostate and hemorrhoid treatment device according to the exemplary embodiment of the present invention will be described in detail with reference to FIGS. 5 to 7.

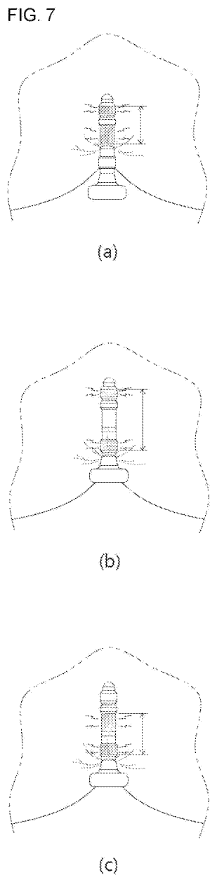

FIG. 5 is a view showing a state in which an anal insertion part is being inserted into the anus of a patient, and FIG. 6 is a view showing a state in which the anal insertion part is inserted into the anus of the patient. In addition, FIG. 7 is a view showing a state in which the anal insertion part is operated after inserted into the anus.

As shown in FIG. 5, the anal insertion part 10 may be inserted into the anus of a human body. The probe module 120 of the anal insertion part 10 may be formed in a cylindrical shape so as not to hurt the anus when inserted thereinto.

As shown in FIG. 6, the operation of the anal insertion part 10 may be controlled by wirelessly communicating with the second wireless communication device 212 of the controller 20 through the first wireless communication device 112.

As shown in FIG. 7, in a state inserted into the anus A, the anal insertion part 10 operates differently according to the control of the controller 20, and generates different stimuli, so as to cure prostate disease and anal disease. As described above, the anal insertion part 10 is operated by the controller 20 and may generate heat through the heating element 124. In addition, the anal insertion part 10 may generate electric stimulation through the upper electrode 121c and the middle electrode 121b, or may generate electric stimulation through the upper electrode 121c and the lower electrode 121a. In addition, electric stimulation may be generated through the middle electrode 121b and the lower electrode 121a.

In this way, electric stimulation of several channels is not applied to a site where the electric stimulation is unnecessary, but gives the electric stimulation only to a specific site, thereby increasing the effect of electrotherapy. In addition, the anal insertion part 10 may generate vibration through the vibrating element 122 when generating electric stimulation. The anal insertion part 10 appropriately massages the anus by generating electric stimulation and vibration together, and promotes smooth blood circulation in the anus.

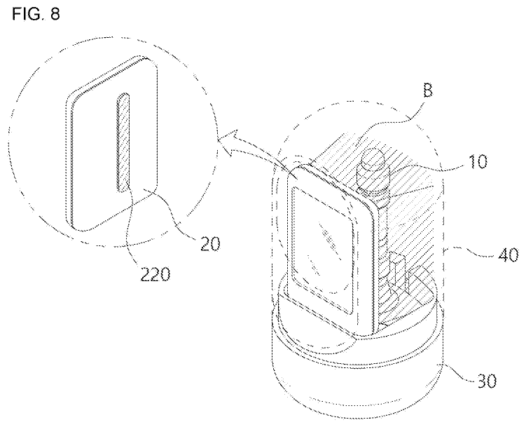

Hereinafter, a state in which the anal insertion part is sterilized will be described with reference to FIG. 8.

FIG. 8 is a view showing a state in which the anal insertion part of FIG. 1 is sterilized by ultraviolet rays.

The controller 20 includes at least one ultraviolet lamp 220 installed on the opposite surface of the outer side surface of the controller 20. Here, as illustrated, one ultraviolet lamp 220 may be provided vertically, or a single or a plurality of ultraviolet lamps 220 may be provided horizontally.

After the controller 20 is set in the sterilization mode, the ultraviolet lamp 220 is inserted into the converter charging part 30, and then is turned on when power is applied thereto through the converter charging part 30, thereby generating ultraviolet rays.

Alternatively, the ultraviolet lamp 220 may be operated when applied with signals, including: a first signal generated when the magnetic sensor is turned on by the interaction between the position magnet of the lid part 40 and the magnetic sensor of the converter charging part 30; and a second signal generated when the second rechargeable battery 211 of the controller 20 is charged.

That is, when the ultraviolet lamp 200 may be operated when the controller 20 is inserted into the accommodation space module 320 of the converter charging part 30 to be charged and the lid part 40 is fastened to the converter charging part 30.

The ultraviolet lamp 220 emits ultraviolet rays B toward the anal insertion part 10 fixed at a distance from the converter charging part 30, and sterilizes the bacteria on the anal insertion part 10. A medium that reflects light is attached to an inner surface of the lid part 40, so as to reflect the ultraviolet rays B output from the ultraviolet lamp 220, whereby the opposite surface of the anal insertion part 10 that does not directly receive the ultraviolet rays from the ultraviolet lamp 220 may also be sterilized.

As described above, when charging the anal insertion part 10 and the controller 20, the anal insertion part 10 is sterilized, and the anal insertion part 10 may be sterilized regularly. In addition, when the charging of the anal insertion part 10 and the controller 20 is completed and the anal insertion part 10 is reused, it is possible to safely reuse the anal insertion part 10.

Although the exemplary embodiments of the present invention have been described above with reference to the accompanying drawings, it will be understood that those skilled in the art to which the present invention pertains may implement the present invention in other specific forms without departing from the technical spirit or essential features thereof. Therefore, the exemplary embodiments described above are to be understood in all respects as illustrative and not restrictive.

1. A prostate and hemorrhoid treatment device comprising:
an anal insertion part comprising
a body module comprising
a first rechargeable battery charged when DC power is applied therein,
a first wireless communication device for wirelessly transmitting and receiving an electric signal, and
an output circuit for outputting a current according to the electric signal output from the first wireless communication device,
a plurality of electrodes provided in a direction perpendicular to the body module and installed apart along a longitudinal direction on an outer circumferential surface to receive the current applied thereto, and
a probe module comprising
a vibrating element installed therein to generate vibration when the current is applied,
a heating element for generating heat, and
a magnet installed at an upper end therein to generate a magnetic force; and
a controller provided with
a second rechargeable battery charged when the DC power is applied therein,
a second wireless communication device capable of performing wireless communication with the first wireless communication device, and
a control module for changing the electric signal output from the second wireless communication device.

2. The prostate and hemorrhoid treatment device of claim 1, further comprising:
a converter charging part comprising
a converter module for converting AC power applied from outside into the DC power and
a plurality of accommodating space modules for vertically accommodating and then fixing the anal insertion part and the controller therein,
wherein the converter charging part charges the first rechargeable battery and the second rechargeable battery by respectively applying the DC power to the anal insertion part and the controller, which are fixed to the accommodation space module.

3. The prostate and hemorrhoid treatment device of claim 2, wherein the converter charging part is coupled to a lid part covering the anal insertion part and the controller, which are fixed to the accommodation space module,
the lid part comprises a position magnet on a lower part surface in contact with the converter charging part, and
the converter charging part comprises a magnetic sensor on an upper part surface in contact with the lid part, so that the converter charging part generates a first signal when the magnetic sensor detects the position magnet and generates a second signal when charging the second rechargeable battery, thereby operating at least one ultraviolet lamp installed on an outer side surface of the controller.

4. The prostate and hemorrhoid treatment device of claim 1, wherein the plurality of electrodes comprises a lower electrode, a middle electrode, and an upper electrode, so that the lower electrode and the middle electrode, or the lower electrode and the upper electrode, or the middle electrode and the upper electrode are able to be operated when the current is applied.

5. The prostate and hemorrhoid treatment device of claim 4, wherein the output circuit comprises a medium-frequency current output circuit for outputting a preset medium-frequency current, and
each of the electrodes comprises a medium-frequency electrode plate through which the medium-frequency current output from the medium-frequency current output circuit is respectively conductive.

6. The prostate and hemorrhoid treatment device of claim 1, wherein the controller comprises a mode selection module having a treatment mode including a prostate treatment mode and a hemorrhoid treatment mode, and
the control module is able to output different electric signals from the second wireless communication device according to the treatment mode selected by the mode selection module.
